(19) United States
(12) Patent Application Publication
(10) Pub. No.: US 2025/0270471 A1
(43) Pub. Date: Aug. 28, 2025

(54) PROCESS FOR PREPARING AGGLOMERATED CRYSTALLINE MEDIUM-CHAIN FATTY ACID SODIUM SALTS

(71) Applicants: Merck Sharp & Dohme LLC, Rahway, NJ (US); Werthenstein Biopharma GmbH, Schachen (CH)

(72) Inventors: Lorenzo Codan, Olten (CH); Eric Sirota, Hoboken, NJ (US); Carlos Pons Siepermann, Hillsborough, NJ (US)

(73) Assignees: Merck Sharp & Dohme LLC, Rahway, NJ (US); Werthenstein Biopharma GmbH, Schachen (CH)

(21) Appl. No.: 18/857,293

(22) PCT Filed: Apr. 19, 2023
(86) PCT No.: PCT/US2023/019026

§ 371 Date: Oct. 16, 2024

Related U.S. Application Data
(60) Provisional application No. 63/495,653, filed on Apr. 12, 2023.
(60) Provisional application No. 63/333,301, filed on Apr. 21, 2022.

(51) Int. Cl. C11C 1/00

(52) U.S. Cl. CPC C11C 1/00

(57) ABSTRACT

The process of the present invention is used to prepare agglomerated, crystalline particles of medium chain fatty acid sodium salts, such as sodium caprate. This process comprises the steps of: a) dissolving a medium chain fatty acid in a first solvent to produce a first solution, wherein the first solvent comprises an aprotic polar solvent; b) adding to the first solution (i) a second solvent, wherein the second solvent comprises a medium chain aliphatic hydrocarbon solvent, and (ii) a solution comprising a sodium salt of a short chain alcohol to create a resulting slurry; and c) isolating agglomerated crystals from the resulting slurry. The first solvent may comprise acetonitrile, and the second solvent may comprise heptane. Further provided are products generated by this process, including sodium caprate products having superior flowability and compression properties.

### BACKGROUND OF THE INVENTION

Sodium salts of medium-length chain (medium chain) saturated organic acids are surprisingly difficult to crystallize. These molecules have a strong propensity towards gelling and making unstirrable batch conditions in most solvent systems. The natural crystalline morphology for these compounds is long fibers/needles that tend to entrain solvent, are difficult to isolate in standard equipment trains, and have undesirable powder properties for formulation manufacturing. Most commercially available material of this kind is either highly expensive and isolated through spray-drying, or retains the suboptimal fiber-like morphology that form from thick unstirrable slurries.

Sodium caprate, or sodium decanoate, is the sodium salt of caproic acid, a 10-carbon saturated fatty acid, which can form micelles and liquid crystalline phases in aqueous solution. Sodium caprate may help the transport of biologically active molecules and, as an FDA-approved food additive and component of finished drug products, it may serve to enhance the bioavailability of an API. In addition, sodium caprate is a known intestinal permeation enhancer. While there are known processes for preparing sodium caprate, including the synthesis described in B. Zacharie, et al., Organic Process Research & Development 2009, 13, 581-583, unlike the present invention, these processes, using any of numerous solvent systems, result in gelling of the sodium caprate material. Gelling makes use of sodium caprate (powder) in manufacturing on large scales difficult and impractical. However, in the few solvent systems in which gelling can be suppressed, sodium caprate crystallizes as small, thin needles, or fibers, which results in unstirrable slurries. Consequently, these slurries present their own challenges, as it is difficult to transfer these from one equipment train to the other, for instance, from the crystallization tank to the filter/drier or the centrifuge. In addition, these particles filter poorly and entrap a significant amount of interstitial liquid, resulting in excessive agglomeration during drying. The cake that is formed requires a significant energy input to be broken up and results in the formation of widely-distributed, hard chunks of material, which impact the ability to formulate the material. These particles are also more vulnerable to cracking, or breakage. In total, sodium caprate is prodigiously difficult to isolate at industrial scale. As a potential consequence of these formulation challenges, there is limited GMP supply of sodium caprate.

As such, there are regulatory and technical barriers to the industrial use of caprate as an excipient in drug product formulations.

### SUMMARY OF THE INVENTION

The instant disclosure provides processes that generate solids that are agglomerates of adjustable particle size, with powder flow properties and compression behavior superior to those of commercially available alternatives. The instant process is cost-effective and generates material with excellent flow and compaction properties, making it suitable for use as an excipient in manufacturing processes and finished drug products. The instant process is also scalable, as it is capable of generating large volumes of solid material. In various embodiments, the instant process comprises a method of producing medium chain fatty acid sodium salts, such as sodium caprate.

Further provided herein are products generated by the instant process. These products—which are solid crystalline powders that may serve as excipients in drug formulations—have superior flow properties and compaction properties relative to commercial medium chain fatty acid sodium salts. These products may be substantially free of gelling or fiber- or needle-like dispersions of crystals.

The present invention is related to a process for preparing agglomerated crystals of a sodium salt of a medium chain fatty acid comprising the steps of:
a) dissolving a medium chain fatty acid in a first solvent to produce a first solution, wherein the first solvent comprises an aprotic polar solvent;
b) adding to the first solution (i) a second solvent, wherein the second solvent comprises a medium chain aliphatic hydrocarbon solvent, and (ii) a solution comprising a sodium salt of a short chain alcohol to create a resulting slurry; and
c) isolating agglomerated crystals from the resulting slurry.

In various aspects, the first solvent comprises or is an aprotic polar solvent. Without wishing to be bound by theory, an aprotic polar solvent is particularly suitable for dissolving a medium chain fatty acid (e.g., capric acid) while avoiding excessive solubilization of the crystalline product (e.g., sodium caprate). In some aspects, the first solvent comprises an aprotic polar solvent selected from acetonitrile, dimethylformamide (DMF), dimethylacetamide (DMAC), and n-methyl-2-pyrolidone (NMP). In some aspects, the second solvent comprises a medium chain aliphatic hydrocarbon solvent selected from heptane, hexane, and octane. In some embodiments, the second solvent comprises a solvent selected from heptane and hexane. As such, in some aspects, the present process comprises the steps of:
a) dissolving a medium chain fatty acid in an aprotic polar solvent selected from acetonitrile, DMF, DMAC, and NMP to produce a first solution;
b) adding to the first solution an aliphatic hydrocarbon solvent selected from heptane and hexane, and about 1 molar equivalent of a solution comprising a sodium salt of a short chain alcohol to create a resulting slurry; and
c) isolating the agglomerated crystals from the resulting slurry.

In some aspects, the present process comprises the steps of:
a) dissolving a medium chain fatty acid in acetonitrile to produce a first solution;
b) adding heptane and about 1 molar equivalent of a solution comprising a sodium salt of a short chain alcohol to the first solution to create a resulting slurry; and
c) isolating the agglomerated crystals from the resulting slurry.

In some embodiments, the sodium salt of a short chain alcohol is sodium methoxide. In some embodiments, the solution comprising a sodium salt of a short chain alcohol comprises methanol or ethanol. In particular embodiments, the solution contains methanol. In particular embodiments, the solution comprising a sodium salt of a short chain alcohol comprises methanol and sodium methoxide.

In various embodiments, the second solvent and solution comprising a sodium salt of a short chain alcohol are added separately, to the first solution. This is referred to in the present disclosure as addition by “separate feed line.”

In various embodiments, the agglomerated crystals in the resulting slurry comprises sodium caprate. This agglomerated crystalline product may be referred to herein as “Product A.”

Other embodiments, aspects and features of the present invention are further described in or will be apparent from the ensuing description, examples and appended claims.

### BRIEF DESCRIPTIONS OF THE DRAWINGS

FIG. 1 shows the X-Ray Powder Diffraction data for Product A using the instant invention, as compared to commercially available alternatives of sodium caprate.

FIG. 2 is a differential scanning calorimeter (DSC) scan of Product A.

FIG. 3 depicts a Thermogravimetric analysis of the Product A.

FIG. 4 depicts the particle size analysis of Product A.

FIG. 5 shows an scanning electron microscope (SEM) image of Product A.

FIG. 6 is a chart depicting the compression performance of a solid formulation of Agglomerated Material (Product A) having one or more compression aids, in comparison to corresponding commercially available forms of crystalline sodium caprate.

FIG. 7 depicts SEM images comparing commercially available forms of sodium caprate crystals to Product A.

FIG. 8 is an SEM image of sodium pelargonate crystals generated in acetonitrile and heptane.

FIG. 9 is an SEM image of sodium laureate crystals generated in acetonitrile and heptane.

FIG. 10. shows light-microscope images depicting the formation of sodium caprate agglomerates using different aprotic polar organic solvents (NMP and DMAC) with heptane.

FIG. 11. shows SEM images of sodium caprate crystals generated in acetonitrile and hexane.

FIG. 12. shows SEM images of sodium caprate crystals generated in acetonitrile and heptane using 1 L/kg (1V) and 2 L/kg (2V) of acetonitrile.

FIG. 13. depicts photographs taken during the performance of an exemplary process used to generate about 1.0 kg of sodium caprate agglomerated crystal product. This product is shown in the rectangular glass dish, at bottom.

### DETAILED DESCRIPTION OF THE INVENTION

The present invention is related to a process for preparing agglomerated crystals of a sodium salt of a medium chain fatty acid comprising the steps of:
a) dissolving a medium chain fatty acid in a first solvent to produce a first solution, wherein the first solvent comprises an aprotic polar solvent selected from acetonitrile, DMF, DMAC, and NMP to produce a first solution;
b) adding to the first solution (i) a second solvent, wherein the second solvent comprises a medium chain aliphatic hydrocarbon solvent selected from heptane, hexane, and octane, and (ii) a solution comprising a sodium salt of a short chain alcohol to create a resulting slurry; and
c) isolating the agglomerated crystals from the resulting slurry.

In a first embodiment, the first solvent of the present process comprises acetonitrile, and the second solvent comprises heptane. Thus, in this embodiment, the present invention is related to a process for preparing agglomerated crystals of a sodium salt of a medium chain fatty acid comprising the steps of:
a) dissolving a medium chain fatty acid in acetonitrile to produce a first solution;
b) adding to the first solution heptane and a solution comprising a sodium salt of a short chain alcohol to create a resulting slurry; and
c) isolating agglomerated crystals from the resulting slurry.

In various aspects, the medium chain fatty acid comprises capric acid, and the first solvent comprises acetonitrile.

In a further aspect of the first embodiment, in step b, heptane and the solution comprising a sodium salt of a short chain alcohol (e.g., sodium methoxide in methanol) are added to the first solution to induce a liquid-liquid phase separation and to create a resulting slurry. In some aspects, the solution comprising a sodium salt of a short chain alcohol comprises 15 wt % to 40 wt % sodium methoxide (e.g., in methanol). In particular aspects, about 1 molar equivalent of a solution comprising 15 wt % to 40 wt % sodium methoxide is added in step b.

In a further aspect of the first embodiment, in step b, heptane and the solution comprising a sodium salt of a short chain alcohol are added to the first solution at a temperature below about 40° C. In some aspects, the heptane is added over about 3.5 to about 4.5 hours at a controlled rate, with constant stirring.

In a second embodiment, the instant invention is directed to a process for preparing agglomerated crystals of sodium caprate (Product A) comprising the steps of:
a) dissolving capric acid in a first solvent to produce a first solution, wherein the first solvent comprises acetonitrile;
b) adding a second solvent and a solution comprising sodium methoxide to the first solution to create a resulting slurry, wherein the second solvent comprises heptane; and
c) isolating the agglomerated crystals of sodium caprate (Product A) from the resulting slurry.

In a further aspect of the second embodiment, the agglomerated crystals are sodium caprate. As such, a process is provided for preparing agglomerated crystals of sodium caprate (Product A) comprises the steps of:
a) dissolving capric acid in acetonitrile to produce a first solution;
b) adding heptane and about 1 molar (e.g., about 0.9 to about 1.5 molar) equivalent of a solution comprising sodium methoxide to the first solution to create a resulting slurry; and
c) isolating the agglomerated crystals of sodium caprate (Product A) from the resulting slurry.

In the second embodiment, the addition of heptane in step b induces a liquid phase-phase separation. In some aspects, the addition of the heptane and the sodium methoxide at a temperature below about 40° C. induces a liquid-liquid phase separation. In some embodiments, about 1.7 L/kg to 2.1 L/kg of heptane is added in this step. In some embodiments, the heptane is added over about 3.5 to about 4.5 hours.

In a further aspect of the first or the second embodiment, after step b, the resulting slurry is stirred for at least one hour. In some aspects, the resulting slurry is stirred for between 1 and 30 hours, 5 and 25 hours, 5 and 15 hours, 10 and 25 hours, 10 and 15 hours, 15 and 25 hours, 20 hours and 25 hours, 25 hours and 30 hours, 20 hours and 30 hours, 20 hours and 24 hours, or 21 hours and 24 hours. In some aspects, the resulting slurry is stirred for about 15 hours, 16 hours, 17 hours, 18 hours, 19 hours, 20 hours, 21 hours, 22 hours, 23 hours, 24 hours, or 25 hours. In some aspects, the resulting slurry is stirred for 15 hours to 25 hours. In particular aspects, the slurry is stirred for between 20 hours and 24 hours.

In a third embodiment, the process for preparing agglomerated crystals of sodium caprate (Product A) comprises the steps of:
a) dissolving capric acid in 1 L/kg to 50 L/kg of acetonitrile to produce a first solution;
b) adding to the first solution 1.5 L/kg to 5 L/kg of heptane and about 0.94 to 1.2 molar equivalent of a solution comprising about 20 wt % to about 40 wt % of sodium methoxide to create a resulting slurry,
c) stirring the resulting slurry for at least one hour; and
d) filtering the resulting slurry to provide agglomerated sodium caprate crystals (Product A).

In some aspects of the above-described embodiments, the addition in step b is performed at a temperature at or below 60° C. In some aspects of the above-described embodiments, the addition in step b is performed at a temperature at or below 50° C., at or below 45° C., at or below 40° C., or at or below 35° C. In some aspects of the above-described embodiments, the addition in step b is performed at a temperature at or below about 40° C. In some aspects, the addition in step b is performed at about 40° C. In some aspects, the addition in step b is performed at about 35° C. In some aspects, the addition in step b is performed at room temperature. In some aspects, the addition in step b is performed at about 22° C. to about 35° C. In some aspects, the addition in step b is performed at about 22° C., 23° C., 24° C., 25° C., 27.5° C., 30° C., 32.5° C., 35° C., 37.5° C., or 40° C.

In some sub-embodiments, the process for preparing agglomerated sodium caprate crystals comprises the steps of:
a) dissolving capric acid in 6 L/kg to 30 L/kg of acetonitrile to produce a first solution;
b) adding to the first solution 1.5 L/kg to 5 L/kg of heptane and 0.75 to 1.5 molar equivalent of a solution comprising about 20 to about 40 wt % of sodium methoxide at a temperature below about 40° C. to induce a liquid-liquid phase separation and create a resulting slurry;
c) stirring the resulting slurry for at least one hour; and
d) filtering the resulting slurry to provide agglomerated sodium caprate crystals.

In a fourth embodiment, the process for preparing agglomerated sodium caprate crystals comprises the steps of:
a) dissolving capric acid in 4 L/kg to 8 L/kg of acetonitrile, to produce a first solution;
b) adding to the first solution about 1.5 L/kg to about 2.5 L/kg of heptane and about 0.96 to 1.05 molar equivalent of a solution comprising about 25 wt % to about 30 wt % sodium methoxide, over about 1.0 to about 10.0 hours, at a temperature of about 22° C. to about 35° C., with constant stirring comprising to produce a resulting slurry;
c) stirring the resulting slurry for at least one hour, optionally between 20 hours and 24 hours; and
d) filtering the resulting slurry to separate resulting solids and drying said resulting solids to provide agglomerated crystals of sodium caprate.

In a fifth embodiment, the process comprises:
a) dissolving capric acid in 6 L/kg to 30 L/kg of acetonitrile, to produce a first solution;
b) adding to the first solution about 1 molar equivalent of a solution comprising about 30 wt % sodium methoxide, over about 4.5 to about 5.5 hours, at a temperature of about 20° C. to about 30° C., with constant stirring;
c) about 1 hour after adding the solution comprising sodium methoxide in step b, adding about 1.5 L/kg to about 4 L/kg of heptane to produce resulting slurry;
d) stirring the resulting slurry for 15 hours to 25 hours; and
e) filtering the resulting slurry to separate resulting solids and drying said resulting solids to provide agglomerated crystals of sodium caprate.

In any of the embodiments, within step d, after filtering the solids that result from the stirring of the slurry, the resulting solids may be washed to remove any residual chemicals (e.g., residual methoxide). In some embodiments, the solids are washed with a solution comprising acetonitrile and methanol. As such, in a sixth embodiment, the process for preparing agglomerated sodium caprate crystals comprises the steps of:
a) dissolving capric acid in 4 L/kg to 8 L/kg of acetonitrile, to produce a first solution;
b) adding to the first solution about 1.5 L/kg to about 2.5 L/kg of heptane and about 0.96 to 1.05 molar equivalent of a solution comprising about 25 wt % to about 30 wt % sodium methoxide, over about 1.0 to about 10.0 hours, at a temperature of about 22° C. to about 35° C., with constant stirring to produce a resulting slurry;
c) stirring the resulting slurry between 20 hours and 24 hours;
d) filtering the resulting slurry to separate resulting solids;
e) washing the resulting solids with a solution comprising acetonitrile and methanol; and
f) drying the resulting solids to provide agglomerated crystals of sodium caprate.

For example, in step e of the sixth embodiment, the resulting solids may be washed with a solution comprising 2 L/kg to 10 L/kg of acetonitrile and methanol. In some sub-embodiments, two washes of 2 L/kg each are performed. In some embodiments, the wash solution comprises about 10:1, 9:1, or 8:1 parts acetonitrile to methanol, by volume. In particular embodiments, the wash solution comprises 9 parts acetonitrile to 1 part methanol (v/v) (9:1). In some embodiments, this washing step is omitted.

In any of the described embodiments, in step a, capric acid is dissolved in 1 L/kg to 50 L/kg of acetonitrile to produce the first solution. In any of the embodiments, capric acid is dissolved in 3 L/kg to 30 L/kg of acetonitrile to produce a first solution. In any of the embodiments, capric acid is dissolved in 6 L/kg to 30 L/kg of acetonitrile to produce a first solution. In any of the embodiments, capric acid is dissolved in 7.5 L/kg to 25 L/kg of acetonitrile to produce a first solution. In any of the embodiments, capric acid is dissolved in 10 L/kg to 25 L/kg of acetonitrile to produce a first solution. In any of the embodiments, capric acid is dissolved in 10 L/kg to 20 L/kg of acetonitrile to produce a first solution. In any of the embodiments, capric acid is dissolved in about 25 L/kg of acetonitrile to produce a first solution. In any of the embodiments, capric acid is dissolved in about 1, 1.5, 2, 2.5, 3, 3.5, 4, 4.5, 5, 5.5, 6, 6.5, 7, 7.5, 8, 8.5, 9, 10, 12, 15, 20, 25, or 30 L/kg of acetonitrile. In some aspects, 1, 2, or 3 L/kg of acetonitrile may be used. In some aspects, about 3 L/kg of acetonitrile is used.

In any of the described embodiments, capric acid is dissolved in 6 L/kg to 8 L/kg of acetonitrile to produce a first solution (i.e., an amount of 6 L/kg to 8 L/kg of acetonitrile is added in step a). In some aspects, about 6.0 L/kg of acetonitrile is used. About 7.0 L/kg or 8.0 L/kg of acetonitrile may be used.

In various aspects, the solution comprising comprising a sodium salt of a short chain alcohol (e.g., sodium methoxide) is added over 1.0 to 10.0 hours, at a temperature of 5° C. to 40° C., with constant stirring. In any of the embodiments, 0.5 to 1.5 molar equivalent of a solution comprising sodium methoxide is added to the first solution. In any of the embodiments, about 0.75 to about 1.5 molar equivalent of a solution comprising 15 wt % to 40 wt %, 20 wt % to 40 wt %, 15 wt % to 35 wt %, about 15 wt % to 30 wt %, or about 25 wt % to 30 wt %, sodium methoxide is added. In some embodiments, about 0.9 to about 1.5, 0.9 to 1.00, about 0.96 to 1.05, or about 0.93 to about 1.00 molar equivalent, of a solution comprising 15 wt % to 40 wt %, or 25 wt % to 30 wt %, sodium methoxide is added. In particular embodiments, 0.97 molar equivalent of 25 wt % to 30 wt % sodium methoxide is added. In some embodiments, the solution comprising comprising a sodium salt of a short chain alcohol (e.g., sodium methoxide) is added over at least 2.0 hours. In any of the embodiments, the sodium methoxide is added over about 4 to about 6 hours, with constant stirring. In some embodiments, the sodium methoxide is added over about 4 hours to about 6 hours, or about 5 to 5.5 hours, at a temperature of about 20° C. to about 30° C. In some embodiments, the sodium methoxide is added over 4.0 hours. In some embodiments, the sodium methoxide is added over about 5 to 5.5 hours.

In any of the embodiments, the second solvent is added to the solution about 1 hour after the solution comprising sodium methoxide is added in step b. In some embodiments, step b comprises adding a second solvent at a controlled rate about 1 hour after sodium methoxide is added, with constant stirring, wherein the second solvent comprises heptane. Step b may comprise adding 1.5 L/kg to 5.0 L/kg of heptane over 3 to 5 hours at a controlled rate, with constant stirring, about 1 hour after adding the solution comprising sodium methoxide. In any of the embodiments, about 1.5 L/kg to about 2.5 L/kg of heptane is added comprising over 3 to 5 hours at a controlled rate, with constant stirring, about 1 hour after sodium methoxide is added. In any of the embodiments, comprising about 1.9 L/kg of heptane is added over 3 to 5 hours at a controlled rate, about an hour after sodium methoxide is added. In any of the embodiments, comprising about 3.5 L/kg to about 4.0 L/kg of heptane is added over 3 to 5 hours at a controlled rate, with constant stirring.

In some embodiments, 1.5 L/kg to 5.0 L/kg, 1.5 L/kg to 4 L/kg, 1.5 L/kg to 2.5 L/kg, 1.7 L/kg to 2.1 L/kg, or 3.5 L/kg to 4.0 L/kg of heptane is added over between about 1.5 and about 5 hours at a controlled rate, with constant stirring. In some embodiments, 1.5 L/kg to 5.0 L/kg, 1.5 L/kg to 4 L/kg, 1.5 L/kg to 2.5 L/kg, or 3.5 L/kg to 4.0 L/kg of heptane is added over 3.5 to 4.5 hours, or over 4.5 to 5.5 hours, at a controlled rate, with constant stirring. In some embodiments, about 1.7 L/kg to 2.1 L/kg of heptane is added over about 3.5 to about 4.5 hours, over about 4.5 to 5 hours, or over about 4.5 to 5.5 hours. In some embodiments, about 3.7 L/kg of heptane is added over about 3.5 to about 4.5 hours, or over about 4.5 to 5 hours. In some embodiments, about 1.9, 2.0, or 2.5 L/kg of heptane is added over over about 4.5 to 5 hours.

In a further aspect of the fourth embodiment, in step c, the heptane is added over about 3.5 to about 4.5 hours at a controlled rate, with constant stirring.

In some embodiments, the second solvent (e.g., heptane) is added to the solution less than 1 hour after adding the solution comprising sodium methoxide. In some embodiments, the second solvent (e.g., heptane) is added to the solution about 10 minutes, 15 minutes, 20 minutes, 30 minutes, 45 minutes, 40 minutes, 50 minutes, or 55 minutes after adding the solution comprising sodium methoxide. In some embodiments, the second solvent is added to the solution approximately simultaneously with the solution comprising sodium methoxide.

The process of the present invention allows for the direct crystallization of agglomerated, crystalline particles of medium chain fatty acid sodium salts, such as sodium caprate. This process avoids the formation of gels and other undesirable processing challenges typically seen with the manufacture of sodium caprate. The instant invention for crystallization of this kind of compound leverages particle agglomeration induced by liquid-liquid phase separation. The instant process may be used to generate production-scale amounts of agglomerated particles of medium chain fatty acid sodium salts, such as sodium caprate. For instance, the instant process may be used to generate weights of sodium caprate that include about 0.5 kg (Example 1A), 1.0 kg (Example 1B), 50 kg, 100 kg, 150 kg, 200 kg, 250 kg, 300 kg, 340 kg, 350 kg, 360 kg, 375 kg, 390 kg, or 400 kg in a single batch. The instant process may be used to generate weights of sodium caprate in two or more batches that include about 800 kg, 900 kg, 1000 kg, or 1100 kg (or 1.1 metric tons).

By identifying a solvent composition that suppresses gel formation and entraps the solids within the dispersed droplets of the second phase, a single-pot, low-energy crystallization process using cheap, commercially accessible starting materials was achieved. The provided agglomerated sodium caprate crystals behave like a traditional slurry and do not entrain solvent, allowing for mild stirring and facile isolation. The agglomerates exhibit sufficient hardness to retain their morphology during discharge and handling. In addition, the provided agglomerates have substantially homogenous morphologies and/or unimodal normal size distribution. The agglomerates also have superior powder flowability. In some embodiments, the agglomerates exhibit a Carr's Index of less than 9.0%. In some embodiments, the agglomerates exhibit a Hausner Ratio of less than 1.15. The Carr's Index and Hausner Ratio metrics correspond to increased flowability. The Carr's Index and Hausner Ratio of any of the disclosed compositions may be calculated using any methods known in the art, such as by measuring the bulk and tapped density of the composition using a graduated cylinder and Tapped Density apparatus.

As such, in some aspects, provided herein are compositions (e.g., crystalline powder compositions) comprising sodium caprate, wherein the composition exhibits a Carr's Index of about less than 20%, less than 15%, less than 14%, less than 13%, less than 12%, less than 11%, less than 10%, less than 9.5%, less than 9%, less than 8.75%, less than 8.5%, less than 8%, less than 7.5%, less than 7%, or less than 6%. In some embodiments, the composition exhibits a Carr's Index of less than 9%. In some embodiments, the Carr's Index is between 8% and 9%. In particular embodiments, the composition exhibits a Carr's Index of 8.7%.

As such, in some aspects, provided herein are compositions (e.g., crystalline powder compositions) comprising sodium caprate, wherein the composition exhibits a Hausner Ratio of about less than 1.25, less than 1.20, less than 1.15, less than 1.14, less than 1.13, less than 1.12, less than 1.11, less than 1.10, less than 1.9, less than 1.8, or less than 1.75. In some embodiments, the composition exhibits a Hausner Ratio of about 1.1. In particular embodiments, the composition exhibits a Hausner Ration of 1.10.

Described herein are products generated by any of the disclosed processes. That is, provided herein are compositions comprising a sodium salt of a medium chain fatty acid (e.g., sodium caprate) generated using any of the disclosed processes. In various embodiments, the products comprise any of the provided agglomerated crystals, such as agglomerated sodium caprate crystals.

### Definitions

Certain technical and scientific terms are specifically defined below. Unless specifically defined elsewhere in this document, all other technical and scientific terms used herein have the meaning commonly understood by one of ordinary skill in the art to which this disclosure relates. That is, terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated.

Any example(s) following the term “e.g.,” or “for example” is not meant to be exhaustive or limiting.

As used herein, and throughout this disclosure, the following terms, unless otherwise indicated, shall be understood to have the following meanings:

As used herein, including the appended claims, the singular forms of words such as “a,” “an,” and “the,” include their corresponding plural references unless the context clearly dictates otherwise.

As used herein, the term “one or more” item each include a single item selected from the list as well as mixtures of two or more items selected from the list.

Unless expressly stated to the contrary, all ranges cited herein are inclusive; i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of +1%, +2%, +3%, +4%, +5%, and +10% and their numerical equivalents. “About” when used to modify a numerically defined parameter (e.g., the temperature, or the length of time for a reaction, as described herein) means that the parameter may vary by as much as 10% below or above the stated numerical value for that parameter; where appropriate, the stated parameter may be rounded to the nearest whole number. For example, a temperature of about 30° C. may vary between 25° C. and 35° C. In addition, the term “or,” as used herein, denotes alternatives that may, where appropriate, be combined; that is, the term “or” includes each listed alternative separately.

As used herein, “Product A” refers to the agglomerated crystalline form of sodium caprate that results from the instant process. In some embodiments, “Product A” is synonymous with “Agglomerated Material.”

As used herein, term “medium chain fatty acid” is intended to mean an aliphatic carbohydrate with a primary carboxylic group and between five and fifteen carbon atoms. Examples include capric acid, lauric acid, pelargonic acid, undecylic acid and the like. In an embodiment of the instant invention, the medium chain fatty acid comprises or is capric acid, pelargonic acid or lauric acid. In further embodiments, the medium chain fatty acid comprises capric acid.

As used herein, term “short chain alcohol” is intended to mean a linear saturated hydrocarbon with one to three carbon atoms and a terminal hydroxide functional group. Examples include methanol or ethanol. In an embodiment of the instant invention, short chain alcohol is methanol. In an embodiment, a sodium salt of a short chain alcohol is sodium methoxide.

As used herein, term “aprotic polar solvent” is intended to mean a compound or mixture of compounds used as a process solvent with a chemical structure that lacks an acidic proton, are polar, and may serve as hydrogen bond acceptors. Examples include dimethylformamide, dimethylacetamide, tetrahydrofuran, or acetonitrile. In an embodiment of the instant invention, the aprotic polar solvent comprises acetonitrile. Any of the described aprotic polar solvents comprise an aprotic polar substance, but may further include additional substances. These additional substances may not necessarily be aprotic or polar themselves.

As used herein, term “medium chain aliphatic hydrocarbon solvent” is intended to mean a compound or a of mixture of compounds used as a process solvent with a chemical structure composed of five to nine carbon atoms connected to form non-aromatic chains and bonded only to each other and hydrogen atoms. Examples include heptane, 2-methylhexane, hexane, octane, and cyclohexane. As used herein, “heptane” may encompass linear heptane, branched heptane, n-heptane, or a blend of heptane isomers (e.g., commercial heptane blends such as “Heptanes, mixture of isomers,” as sold by Thermo Scientific Chemicals). As used herein, “hexane” may encompass linear hexane, branched hexane, n-hexane, or a blend of hexane isomers. In an embodiment of the instant invention, the medium chain aliphatic hydrocarbon solvent comprises n-heptane (which is referred to in the Examples as simply “heptane”). In another embodiment of the instant invention, the medium chain aliphatic hydrocarbon solvent is n-hexane. Any of the described medium chain aliphatic hydrocarbon solvents comprise a medium chain aliphatic hydrocarbon, but may further include additional substances. These additional substances may not necessarily be medium chain aliphatic hydrocarbons themselves.

As used herein, the phrase “controlled rate” is intended to mean an addition of solution using flow rates planned prior to the start of the batch, typically delivered using a pump or flow controller and dosed following a program or schedule.

As used herein, the phrase “constant stirring” is intended to mean a stirring of the solution substantially without interruption. This phrase encompasses the occurrence of one or more interruptions to the stirring, these interruptions collectively having insubstantial impact to the production of an intended slurry (e.g., a few interruptions of 1-3 seconds each). Constant stirring may be performed mechanically (e.g. by magnetic stir bar) or manually.

The instant processes may use or comprise one or more steps of agitation of a solution. Examples of agitation techniques include, but are not limited to, overhead stirring, magnetic stirring, shaker plate mixing and the like.

A “liquid-liquid phase separation” (LLPS) occurs when a liquid mixture of two solutions contains solvents that are immiscible and is caused by a thermodynamically non-ideal solution behavior of the solvents involved. The Gibbs free energy is a thermodynamic potential that is minimized when a system reaches equilibrium. If the solvent mixture behaves ideally, the Gibbs free energy has one local minimum and no phase separation occurs. If the solvent mixture's behavior is in turn non-ideal, a temperature and pressure range may exist where the Gibbs free energy has two local minima, which can cause two thermodynamically stable liquid phases, with different overall composition, to form. For example, methanol and acetonitrile are completely miscible solvents, while heptane has a miscibility gap with both methanol and acetonitrile. For the two solvent systems consisting of, or comprising, methanol and heptane and of acetonitrile and heptane, respectively, a set of compositions exists, in which the solvents will not mix resulting in the formation of two immiscible liquid phases. A miscibility gap exists also for the ternary solvent system consisting of, or comprising, methanol, acetonitrile and heptane, i.e., a set of compositions exists, in which the three solvents will not mix resulting in the formation of two immiscible liquid phases. The formation of two immiscible liquid phases upon addition of a solvent is herein referred to as liquid-liquid separation (LLPS).

This instant invention uses known filtering techniques, including but not limited to centrifugation, vacuum filtration, pressure filtration and the like.

Methods for preparing the agglomerated crystals of a sodium salt of a medium chain fatty acid, particularly crystals of sodium caprate, are illustrated in the following Schemes and Examples Starting materials are made according to procedures known in the art or as illustrated herein. The following abbreviations are used herein:
DMAC dimethylacetamide
DMF dimethylformamide
DSC Differential Scanning calorimeter
FID Flame Ionization Detector
GC Gas Chromatography
GMP Good Manufacturing Procedures
IPA Isopropyl Alcohol
LOQ Limit of Quantitation
Me methyl
MeCN acetonitrile
MeOH methanol
NMP n-methyl-2-pyrrolidone
RI Refractive Index
rt room temperature
TGA Thermogravimetric analysis
v/v volume/volume;
XRPD X-ray Powder Diffraction

In some cases, the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLES

### Example 1A

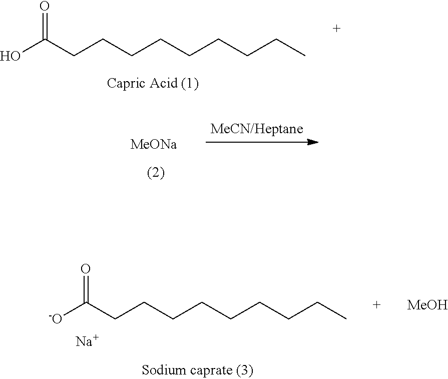

Capric acid (1) (25 g, 145 mmol) and acetonitrile (630 ml) were combined in an appropriate vessel with a suitable agitator. The batch was stirred at room temperature until complete dissolution was achieved. Sodium methoxide (2) (8.23 g, 152 mmol) was added as a 25 wt % solution in methanol (32.92 g) over a period of 5 hours under vigorous agitation, forming a slurry. After the first hour of the addition of (2) had elapsed, heptane (93-103 ml) was concurrently added over a period of four hours using a separate feed line. The resulting slurry was stirred for an additional hour. Solids were filtered, washed with acetonitrile (100 ml twice), then dried under vacuum with a nitrogen sweep at 35-40° C. to afford crystalline sodium caprate (3, Product A) (27.79 g, 99% yield).

### Example 1B

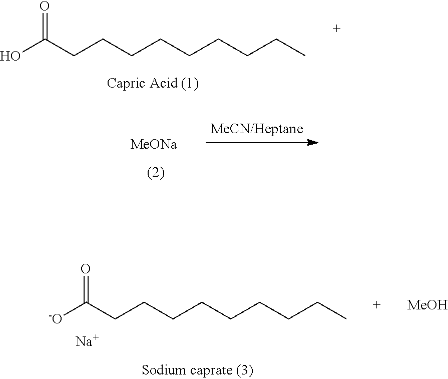

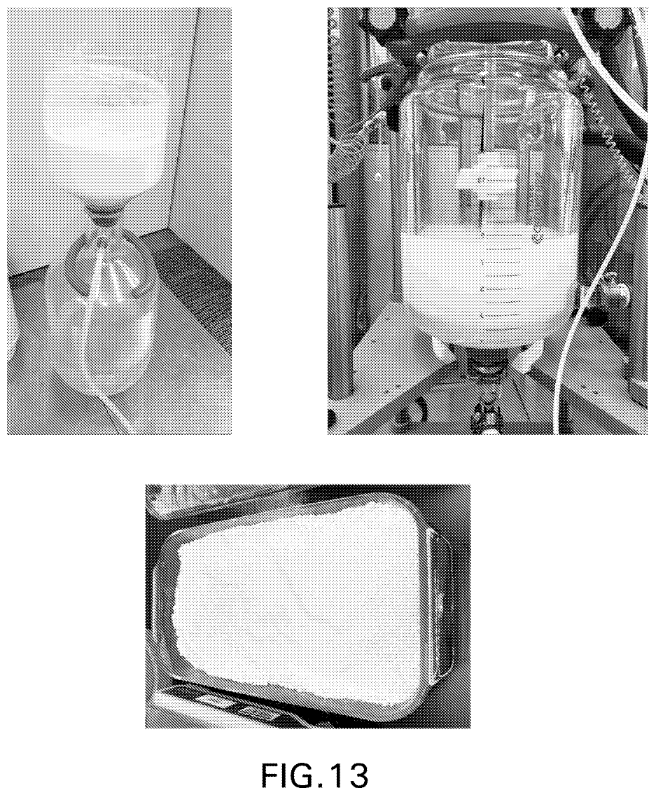

Capric acid (1) (1.00 kg, 5.81 mol) and acetonitrile (6.0 L) were combined in an appropriate vessel with a suitable agitator. The batch was stirred at room temperature until complete dissolution was achieved. Sodium methoxide (2) (0.304 g, 5.63 mol) was added as a 30 wt % solution in methanol (1.01 kg) over a period of 5.5 hours under vigorous agitation. Starting simultaneously with the addition of (2), heptane (1.9 L) was concurrently added over a period of 5 hours using a separate feed line. The resulting slurry was stirred for an additional 24 hours. Solids were filtered, washed with a solution of acetonitrile:methanol (9:1 by volume), 2 L twice, then dried under vacuum with a nitrogen sweep at 35-40° C. to afford crystalline sodium caprate (3, Product A) (1.09 kg, 97% yield). Photographs of the slurry and isolated sodium caprate crystalline material generated using this procedure is shown in FIG. 13. A 1.0 kg scale of product was generated.

### Example 1C

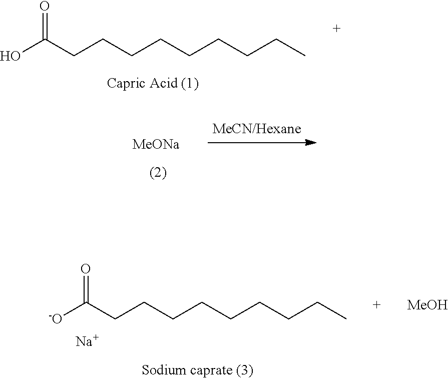

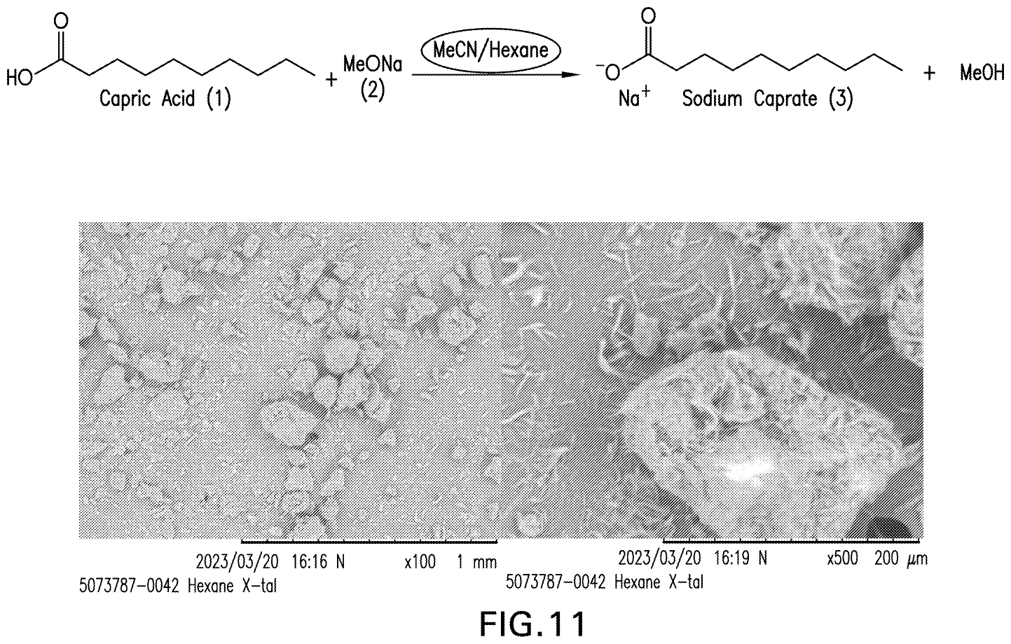

Capric acid (1) (5.02 g, 29.1 mmol) and acetonitrile (30 ml) were combined in an appropriate vessel with a suitable agitator, a homogeneous solution develops. Charged sodium methoxide (2) (28.3 mmol) as a 30 wt % solution in methanol over a period of 5 hours under vigorous agitation, forming a slurry. Starting simultaneously with the addition of (2), hexane (10.54 ml) was concurrently added over a period of 5 hours using a separate feed line. The resulting slurry was stirred for an additional 17 hours. Solids were then filtered, washed with a solution of acetonitrile:methanol (9:1 by volume), 15 ml twice, then dried under vacuum with a nitrogen overhead sweep at 40° C. to afford crystalline sodium caprate (3, Product A) (5.24 g, 93% yield). FIG. 11 depicts material generated using this procedure.

### Example 1D

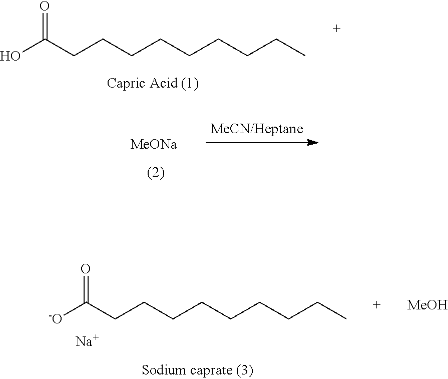

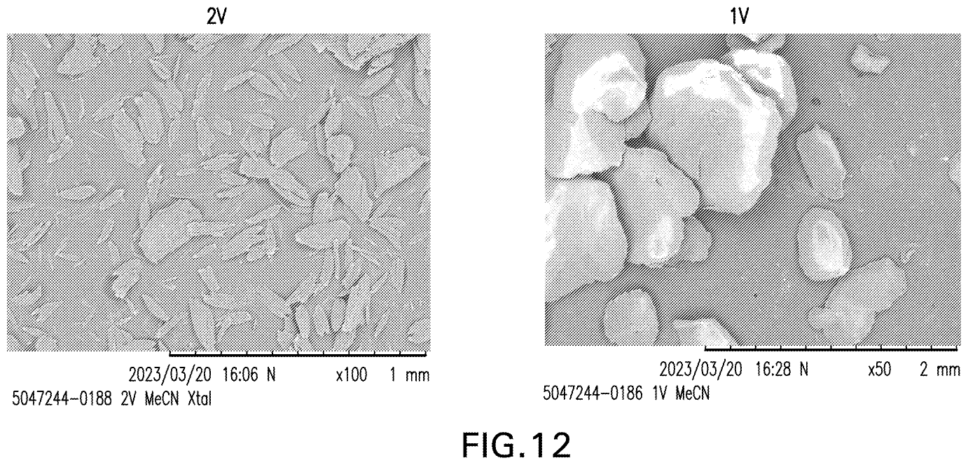

Capric acid (1) (5 g, 29 mmol) and acetonitrile (10 ml) were combined in an appropriate vessel with a suitable agitator. The batch was stirred at 35° C. until complete dissolution was achieved. Sodium methoxide (2) (1.55 g, 29 mmol) was added as a 30 wt % solution in methanol (5.18 g) over a period of 5.5 hours under vigorous agitation, forming a slurry. Simultaneously to the addition of (2), heptane (7.4 ml) was concurrently added over a period of 5 hours using a separate feed line. The resulting slurry was stirred for an additional 15 hours. After the age, an additional 1 ml of heptane was added and the batch heated to 40° C. The slurry was aged for an additional 3 hours. Solids were filtered, washed with acetonitrile (10 ml twice), then dried under vacuum with a nitrogen sweep at 35-40° C. to afford crystalline sodium caprate (3, Product A) (4.1 g, 74% yield). FIG. 12 depicts material made using this procedure.

### Example 1E

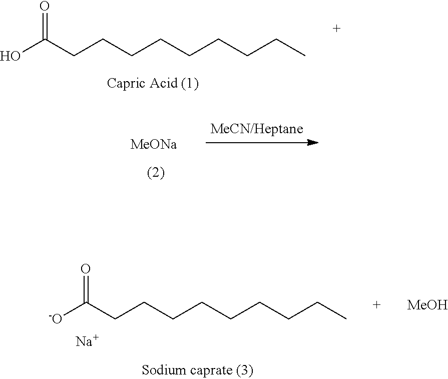

Capric acid (1) (10 g, 58.5 mmol) and acetonitrile (10 ml) were combined in an appropriate vessel with a suitable agitator. The batch was stirred at 40° C. until complete dissolution was achieved. Sodium methoxide (2) (3.17 g, 58.5 mmol) was added as a 30 wt % solution in methanol (10.57 g) over a period of 5.5 hours under vigorous agitation, forming a slurry. Simultaneously to the addition of (2), heptane (15.15 ml) was concurrently added over a period of 5 hours using a separate feed line. The resulting slurry was stirred for an additional 24 hours. Solids were filtered, washed with a solution of acetonitrile:methanol (9:1 by volume), 20 ml twice, then dried under vacuum with a nitrogen sweep at 35-40° C. to afford crystalline sodium caprate (3, Product A) (9.48 g, 83% yield). FIG. 12 depicts material made using this procedure.

### X-Ray Powder Diffraction (XRPD)

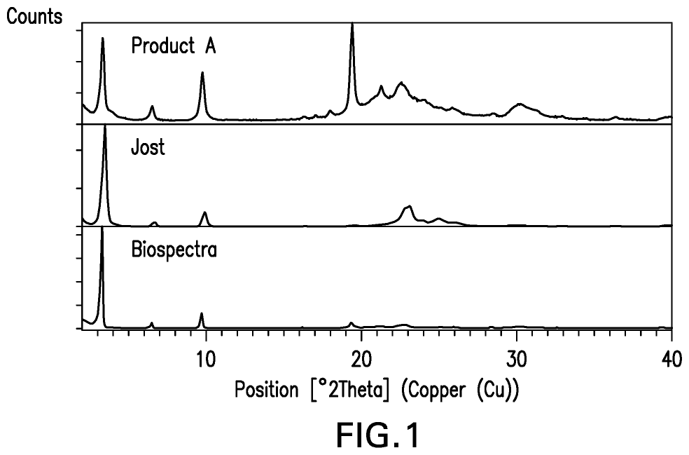

X-ray Powder Diffraction (XRPD) data, as seen in FIG. 1, were acquired on a Panalaytical X-Pert configured in the Bragg-Brentano configuration and equipped with a Cu radiation source with monochromatization to Kα achieved using a Nickel filter. A fixed slit optical configuration was employed for data acquisition. Data were acquired between 2 and 40° 20. Samples were prepared by gently pressing the sample onto a zero background silicon holder. All samples presented in FIG. 1 were obtained in this manner, for comparisons of material made within the instant process and commercial material.

FIG. 1 depicts the XRPD pattern for Product A, the material made by the instant process, and shows a stacked comparison with the XPRD patterns of commercially available alternatives of sodium caprate (such as those obtained from Jost and BSI) made by different processes. FIG. 1 demonstrates there are differences in the intensity of the reflections, indicating that the preferred phases generated vary, but the overall “fingerprint” of the crystal patterns is the same. The instant process generated sodium caprate crystals with a vastly different morphology than commercially available embodiments made by more costly procedures.

Differential Scanning calorimeter (DSC)

A TA Instruments Discovery Differential Scanning calorimeter (DSC) was used to monitor the thermal events as a function of temperature increase. Samples of Product A (2-5 mg) in closed non-hermetic aluminum pans with 2-pinholes were cycled twice from 10 to 300° C. at a heating rate of 10° C./min.

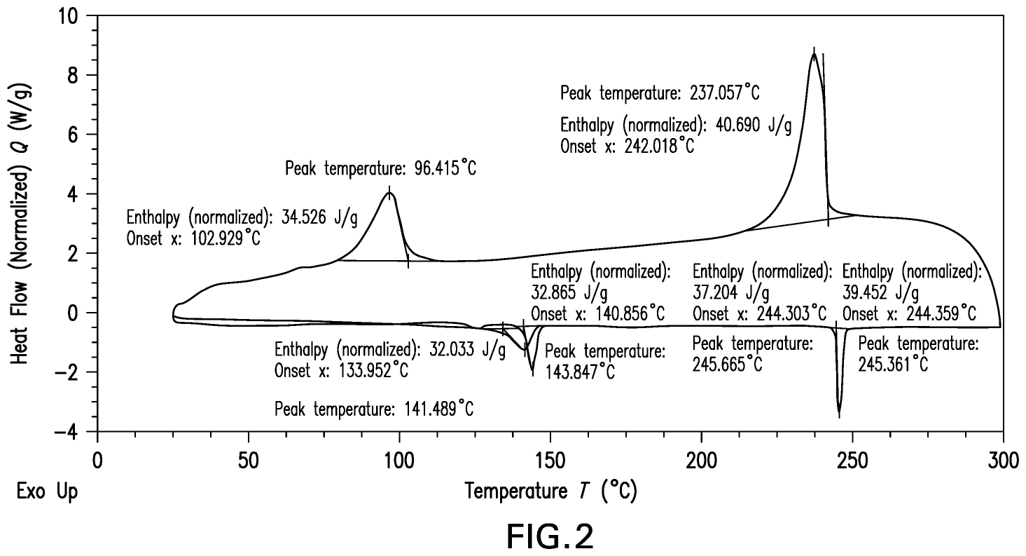

The image of FIG. 2 shows two heat cycles and one cooling cycle performed on the sodium caprate material from room temperature up to 300° C. The down-facing peaks are endotherms, showing that the material is absorbing heat, which suggests a change in the crystal/solid state, or a phase change (e.g., melting or boiling). The curve on the top is what happens to the same sample as it is cooling. During cooling, there are upward peaks (exotherms, where heat is released). These are the reversals of the physical phenomena that took place during the heating. The presence of a hysteresis between the onset and reversal temperatures typically occurs due to differences in kinetic barriers between the forward and backward processes. The width of the hysteresis is usually dependent on the rate at which the temperature is being changed during the DSC scan. There are two heating curves (below zero in the y axis) overlayed in FIG. 2. The overlap of the two lines demonstrates that the changes experienced by Product A during the experiment were reversible and Product A was not destroyed during the scan. The change in width of the first downward peaks between the two heating curves is related to the presence of some absorbed water in the initial scan. The DSC pattern can be used as a characterization technique because the position and area of the peaks, and the overall shape of the scan are specific to sodium caprate.

### Residual Solvent by Gas Chromatography (GC)

Standard Preparation: By serial dilution prepare a 0.01% v/v standard of n-heptane, methanol and acetonitrile in diluent for quantitation and a 0.001% v/v Limit of Quantitation (LOQ) for limit reporting.

Sample Preparation: ˜ 20 mg/mL sample dissolved in diluent. Vortex and sonicate as needed to dissolve sample.

### Instrument Conditions:

| Instrument | Agilent Technologies 6890N | |
|---|---|---|
| Detector | FID | |
| Column | Agilent DB-Wax UI, 30 m × 0.25 mm, 0.5 μm film | |
| | thickness, or equivalent (e.g.: Agilent Column | |
| | (Part# 122-7033UI) | |
| Injection | 0.5 | μL |
| Volume | | |
| Split Ratio | 20:1 | |
| Inlet | 180° | C. |
| Temperature | | |
| Detector | 250° | C. |
| Temperature | | |
| Carrier Gas | Helium or Hydrogen (minimal shifts in retention | |
| | expected depending on carrier gas type) | |
| Carrier Gas | 2.0 mL/min (constant flow) | |
| Flow Rate | | |
| Diluent | 80:20% v/v/IPA:H2O | |
| Run Time | 10 | min |

| | Single ramp level | | | | | |
|---|---|---|---|---|---|---|
| | | | Initial | | | Final |
| | | Initial | Hold | Ramp Rate | Final | Hold |
| | | Temperature | Time | (° C./ | Temperature | Time |
| | Level | (° C.) | (minutes) | minute) | (° C.) | (minutes) |
| Oven | 1 | 40 | 2.0 | 25 | 230 | 0.4 |
| Temperature | | | | | | |
| Program | | | | | | |

### Results:

| Solvent | n-Heptane | Methanol | Acetonitrile |
|---|---|---|---|
| Volume % | Not Detected | 0.04 | <LOQ |

LOQ (limit of quantitation): 393 ppm for acetonitrile

### Thermogravimetric Analysis

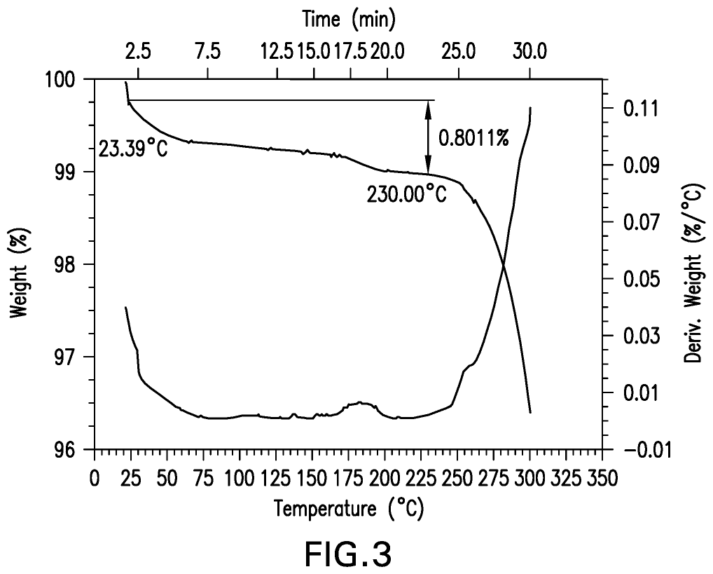

Thermogravimetric analysis (TGA) of Product A was carried out on a TA Q 500 Thermogravimetric Analyzer (TA Instrument). Samples (5-15 mg) in were heated from 25 to 320° C. at 10° C./min, with a nitrogen purge of 200 mL/min. As seen in FIG. 3, the curve at the top of FIG. 3 monitored the change in mass of Product A as it was heated under a nitrogen atmosphere. The gentle mass loss (0.8 wt %) in the first 250° C. matched well with the expected absorbed surface water in the material. The onset of a drop-off after 250° C. indicated the beginning of decomposition or evaporation. The curve at the bottom of the graph corresponds to the derivative of the change in mass, capturing the rate of change of the top curve.

### Particle Size Analysis by Microtrac FlowSync

Instrument make and model: Microtrac, M5001-3L Sync+FlowSync

Method: Approximately 50 mg powder sample was transferred into a 20 mL scintillation vial and 5 mL of IsoparG/0.25% w/v lecithin fluid was added into the vial followed by gentle shaking to disperse the particles. After instrument initialization and background measurement (30 seconds), the suspension was poured into the flow cell unit. The vial was triple-rinsed with 1 mL of IsoparG/0.25% Lecithin fluid (total 3 mL) and all the rinsate was poured into the FlowSync. Measurement parameters included volume distribution, geometric 8 root progression between 0.0215 and 2000 μm, residuals disabled, standard filter enabled, particle RI=1.51; irregular shape; fluid RI=1.42, flow 60%. Particle size distributions were calculated as the average of three 30 second scans. The results were reported as volume distributions. Samples were analyzed without sonication, 30 seconds, 60 seconds and 90 seconds sonication powder of 25%.

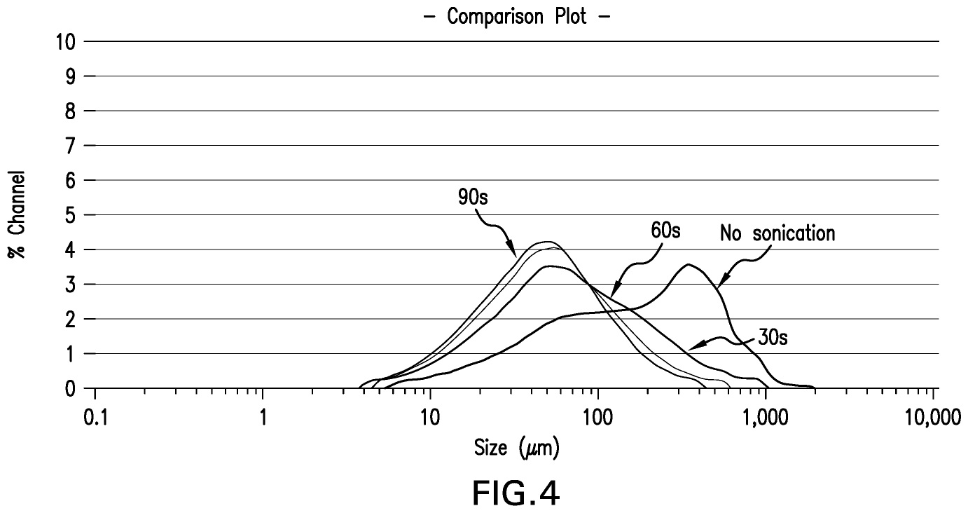

Sonication is a standard laboratory technique in which vibrational energy is applied to the powder to help disperse lumps within the material and ensure that the particle size measurements capture the true size of the product particles. Lumping is commonly observed for dry solids due to natural adhesion and can skew measurements to overestimate the size of powder particles, thus inducing sufficient sonication can be important for analytical accuracy. FIG. 4 shows a representative volume-weighted particle size distribution results for a batch of Product A made with the process described in Example 1. The curves show the probability density of a particle of the product to have a radius of the size specified in the x-axis. The curves also show the impact of sonication on the measured particle size. The reduction and normalization of particle size with increased sonication was typical behavior that showed de-agglomeration of the dry solids towards the “true” distribution of primary particles, which was best captured by the curves labelled for 60 s and 90 s. FIG. 4 shows that the particle size distribution is the same at 60 s and 90 s of sonication, indicating that samples of this material need at least 60 s for adequate measurement.

FIG. 4 also demonstrates how the instant process generated primary particles with a unimodal normal size distribution, which are desirable for manufacturing processes. Unimodal distributions are desirable since they indicate uniformity of the particles, with minimal fines or large agglomerates, which would lead to uneven flow, filtration, and compression behavior. Uniform distributions are also an indication of proper control during the crystallization and agglomeration processes, since they provide evidence that undesirable particle-generating phenomena, such as attrition, are not taking place, and the overall particle size and morphology are set by the controlled variables manipulated during batch design.

### Scanning Electron Microscope (SEM)

Sodium caprate powder samples were mounted on SEM stubs (32 mm) using a carbon sticky. The samples were sputter coated with platinum. The samples were loaded into the Hitachi TM3030 Tabletop Scanning Electron Microscope. The samples were imaged in high vacuum mode and images were acquired using the secondary electron (SE) detector. The voltage was set to 2 kV and the Spot Intensity was set to 30 (unities). Images were acquired at several magnifications.

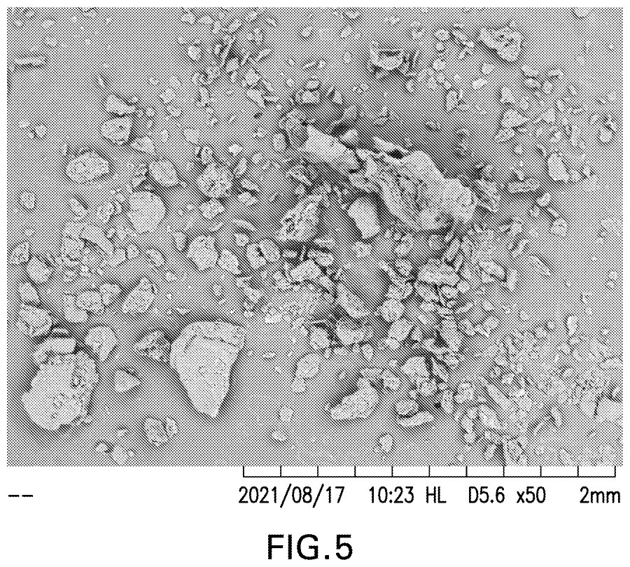

Imaging in FIG. 5 demonstrated the morphology of Product A existed as well-defined agglomerated plate-like primary particles. Such a morphology is extremely difficult to achieve without spray drying and is desirable over elongates plates or needles. The demonstrated morphology reflects superior compression performance in manufacturing procedures and dosage forms. that use Product A as an excipient.

### Compression Performance

Samples of (i) sodium caprate powder as generated by the instant process and (ii) formulations containing sodium caprate samples were compressed into cylindrical compacts using a single-station compaction simulator. Samples were compressed into compacts using a 9.525 mm round flat-face tablet press tooling using a range of compression pressures between 10 and 400 Mega Pascals (MPa). Compaction simulation of the formulations were performed under force control. Combined punch velocities were ranged between 50 and 100 mm/s. Weight (W), thickness (h), diameter (D) and hardness (B) of the resulting compacts were measured. Hardness is defined as the peak force needed to fracture the cylindrical compact. Compact weights were controlled between 225 and 375 mg. The resulting values for thickness and hardness were used to compute the tablet tensile strength (T) using Equation X. The resulting tablet tensile strength and compression pressures are combined into a tabletability curve.

$$T = \frac{2 B}{π hD} \qquad Equation X$$

Table 1 shows the composition (makeup) of tested example formulated tablet products containing sodium caprate from different commercial sources. These formulations contain a disintegrant, a glidant, a lubricant, and two compression aids (i.e., diluents or binders): lactose and microcrystalline cellulose. Four of these formulations contained commercially available sodium caprate lots, and one used Agglomerated Material Product A generated by the instant process. Formulation containing Commercial Material 1 used sodium caprate from Pfaltz & Bauer, Formulation containing Commercial Material 2 used sodium caprate from BSI, Formulation containing Commercial Material 3 used sodium caprate from TCI Chemicals, and Formulation containing Commercial Material 4 used sodium caprate from Jost.

TABLE 1
Composition of Formulated Products Containing
Sodium Caprate from Different Sources

| Compound | Function | wt/wt % |
|---|---|---|
| Sodium Caprate | Permeation enhancer | 36.18 |
| Microcrystalline cellulose | Compression aid | 29.65 |
| Lactose | Compression aid | 29.65 |
| Sodium Croscarmellose | Disintegrant | 3.015 |
| Silicon Dioxide | Glidant | 1.005 |
| Magnesium Stearate | Lubricant | 0.5025 |

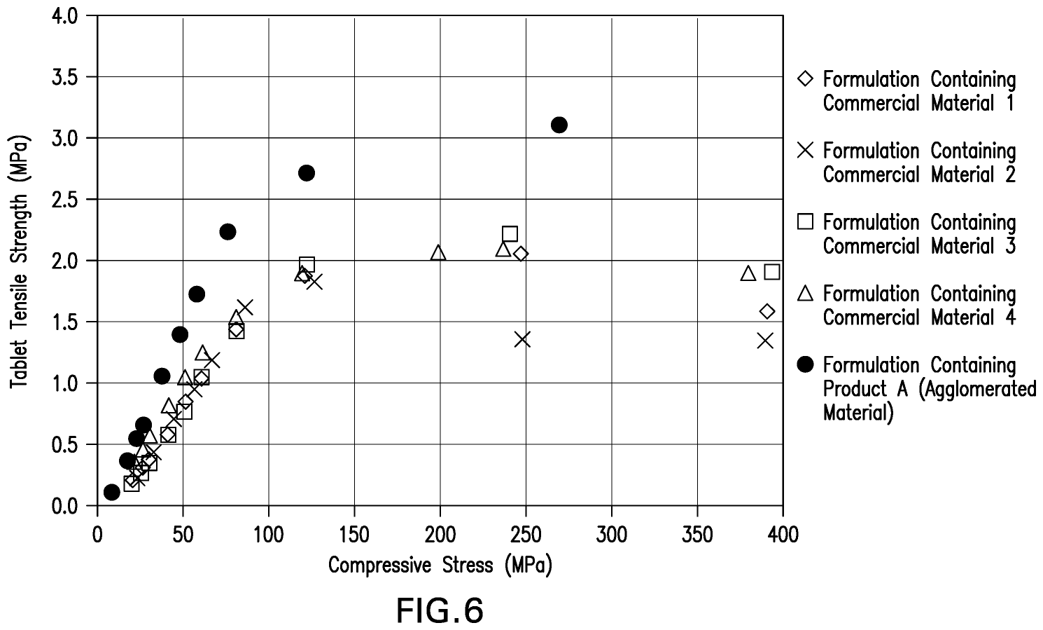

The plot in FIG. 6 shows the compaction profiles results of the five formulations. These results show that Product A (solid circles) exhibited superior compression performance when compared to all tested commercially available materials. FIG. 6 reflects the tensile strength of a tablet formulated with sources of sodium caprate (including Product A). The image shows that the materials produced with Product A (depicted as solid circles) yielded the toughest material within a relevant processing range of compression pressures. The in-die powder bulk densities of these five formulations were estimated using the compact mass and the fill die-volume before compression. The in-die bulk density for Formulation containing Commercial Material 2 was the lowest of all tested materials, nominally 0.39 g/mL. The in-die bulk density of the remaining four formulations ranged between 0.44 and 0.47 g/mL. All observed in-die bulk densities show acceptable values for processing these blends through tabletting.

### Flowability

The flow properties of Product A and sodium caprate materials from different commercial sources were compared using bulk and tapped density measurements. Bulk and tapped densities refer to the density with which the powder packs onto itself. Low densities are a sign of material that will not flow or pack well. The Carr's Index and Hausner ratio are calculated metrics using the measures of bulk and tapped density. Low values for both metrics indicate that materials have improved flow properties. The bulk density was measured using a 100 mL graduated cylinder and no less than 50 mL of powder material. The powder material was dispensed into the graduated cylinder and both the mass and volume of material were recorded. Calculating the ratio of mass and volume yielded the resulting bulk density of the powder. The graduated cylinder was then tapped for 1,250 events using a Tapped Density apparatus following USP <661>. The resulting volume of the material was then used to compute the resulting tapped density value.

Table 2 reports the bulk and tapped density of the material and provides the results for the calculated Carr's Index and Hausner Ratio. As shown in Table 2, Product A exhibits a Carr's Index of 8.7% and a Hausner Ratio of 1.10. Both of these values are significantly lower than those of Commercial Materials 1-4, indicating that Product A exhibits superior powder flowability. In spite of its low density, the agglomerated sodium caprate crystals produced by in this invention (Product A) has excellent flow properties, which are reflected by both its low Carr's Index and Hausner Ratio.

TABLE 2
Flowability Measurement of Sodium
Caprate from Different Sources

| | Bulk | Tap | Carr's | |
|---|---|---|---|---|
| Formulation | Density | Density | Index | Hausner |
| Containing: | (g/mL) | (g/mL) | (%) | Ratio |
| Commercial Material 1 | 0.38 | 0.58 | 34 | 1.53 |
| Commercial Material 2 | 0.19 | 0.36 | 47 | 1.89 |
| Commercial Material 3 | 0.40 | 0.58 | 31 | 1.45 |
| Commercial Material 4 | 0.52 | 0.69 | 25 | 1.33 |
| Product A | 0.21 | 0.23 | 8.7 | 1.10 |

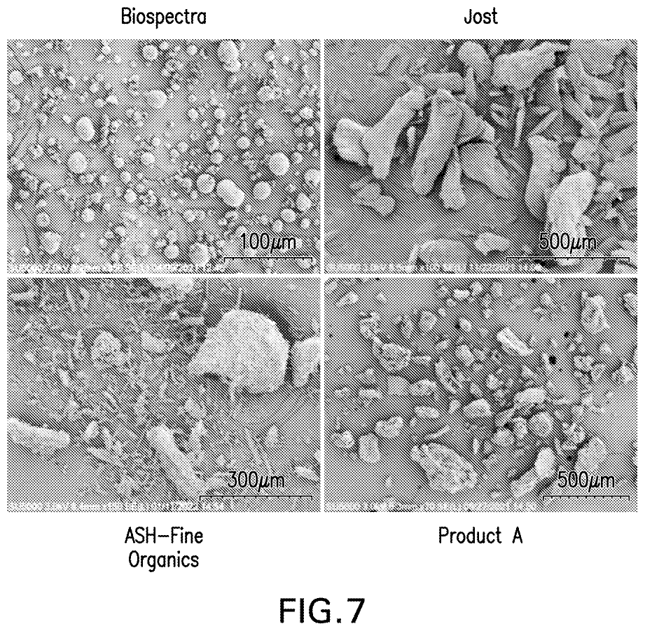

FIG. 7 shows SEM images of sodium caprate from Commercial Materials 1-4, providing a comparison of their respective morphologies. The method of preparation for this compound has significant impact on the structure and physical appearance of the material. Commercial Material 1 (sodium caprate from BSI) was smooth spheres, made through spray-drying. Commercial Material 3 (sodium caprate from TCI Chemical) and Commercial Material 4 (sodium caprate from Jost) were large elongated, thin plates. Product A constituted an agglomerated solid, with a rough surface area and no elongation in any axis. The instant process enables this unique morphology of Product A, which is amenable to manufacturing and handling in standard equipment and has desirable properties for formulation manufacturing.

### Example 1F

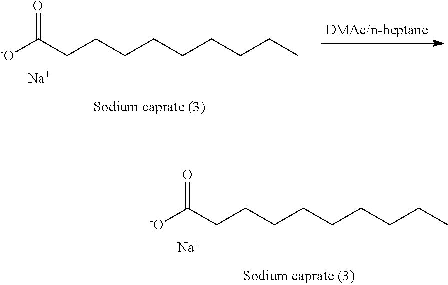

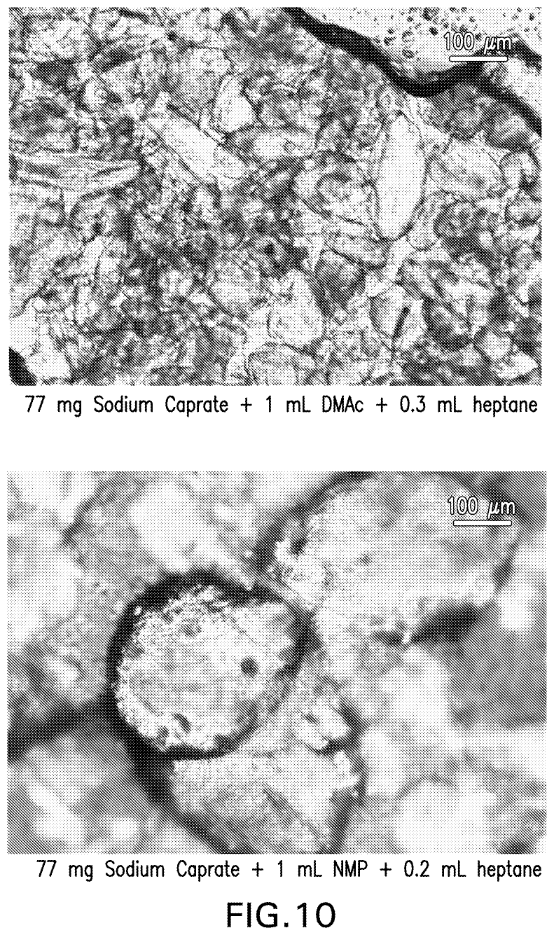

Sodium Caprate (3) (77 mg, 0.40 mmol) and dimethylacetamide (DMAc) (1 ml) were combined in a 4 mL vial to form a slurry. Charged n-heptane (0.3 mL) to the vial, forming a slurry of agglomerated particles. A light microscope image depicting these particles is shown in FIG. 10, top panel.

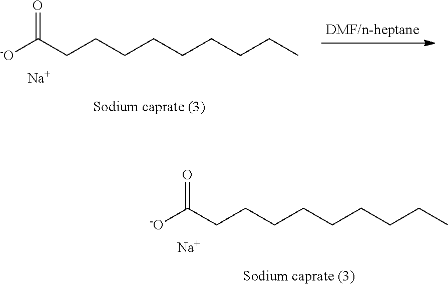

Sodium Caprate (3) (78 mg, 0.40 mmol) and dimethylformamide (DMF) (1 ml) were combined in a 4 mL vial to form a slurry. Charged n-heptane (0.2 mL) to the vial, forming a slurry of agglomerated particles.

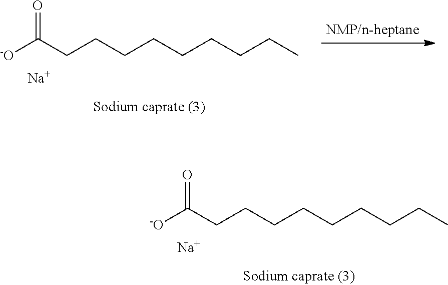

Sodium Caprate (3) (88 mg, 0.45 mmol) and n-methyl-2-pyrrolidone (NMP) (1 ml) were combined in a 4 mL vial to form a slurry. Charged n-heptane (0.2 mL) to the vial, forming a slurry of agglomerated particles. A light microscope image depicting these particles is shown in FIG. 10, bottom panel.

Each of DMAc, DMF, and NMP are polar aprotic solvents. As underscored by the images shown in FIG. 10, a combination of sodium caprate with heptane and a polar aprotic solvent yields agglomerated crystalline sodium caprate product. As such, any of several polar aprotic solvents may be used in the disclosed process of manufacturing sodium caprate agglomerates.

### Example 2

### Preparation of Sodium Pelargonate (C9)

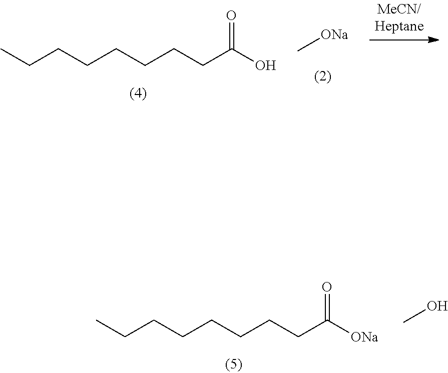

Pelargonic acid (4) (2.51 g, 15.9 mmol) and acetonitrile (63.5 ml) were combined in an appropriate vessel with a suitable agitator, resulting in a homogeneous solution. The solution was charged with sodium methoxide (2) (15.9 mmol) as a 25 wt % solution in methanol over a period of 5 hours under vigorous agitation, forming a slurry. After the first hour of the addition of (2) had elapsed, the solution was charged with heptane (9.3 ml) concurrently over a period of four hours. The slurry was stirred for an additional hour. Solids were filtered, washed with acetonitrile (15 ml twice), then dried under vacuum with a nitrogen sweep at 35-40° C. to afford sodium pelargonate (5) (2.68 g, 94% yield).

### Scanning Electron Microscope (SEM)

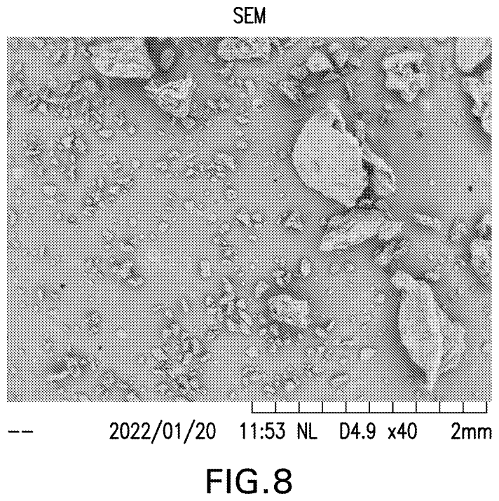

Sodium pelargonate powder samples were mounted on SEM stubs (32 mm) using a carbon sticky. The samples were sputter coated with platinum. The samples were loaded into the Hitachi TM3030 Tabletop Scanning Electron Microscope. The samples were imaged in high vacuum mode and images were acquired using the secondary electron (SE) detector. The voltage was set to 2 kV and the Spot Intensity was set to 30 (unities). Images were acquired at several magnifications. Imaging in FIG. 8 demonstrated the morphology of the agglomerated crystals of sodium pelargonate existed as well-defined plate-like primary particles. Such a morphology is extremely difficult to achieve without spray drying and is desirable over elongates, plates or needles because it tends to display superior compression performance in manufacturing procedures.

### Example 3

### Preparation of Sodium Laureate (C12)

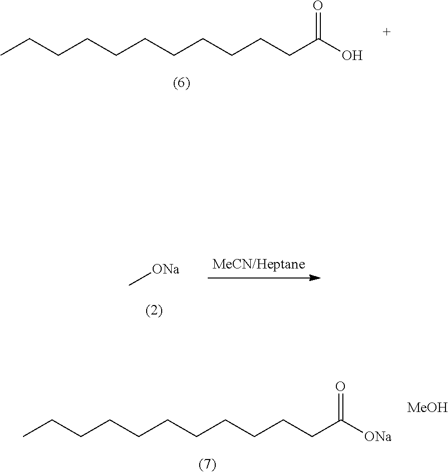

Lauric acid (6) (2.5 g, 12.5 mmol) and acetonitrile (63.3 ml) were combined in an appropriate vessel with a suitable agitator. The batch was stirred at 30° C. until complete dissolution was achieved then cooled to room temperature. The solution was charged with sodium methoxide (2) (12.48 mmol) as a 25 wt % solution in methanol over a period of 5 hours under vigorous agitation, forming a slurry. After the first hour of the addition of (2) had elapsed, the solution was charged with heptane (9.3 ml) concurrently over a period of four hours. The slurry was stirred for an additional hour. Solids were filtered, washed with acetonitrile (15 ml twice), then dried under vacuum with a nitrogen sweep at 35-40° C. to afford sodium laureate (7) (2.7 g, 97% yield).

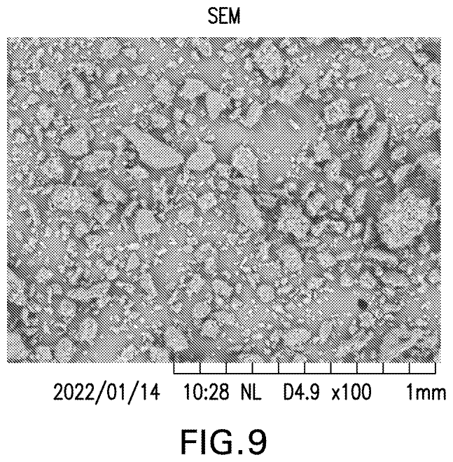

Scanning Electron Microscope (SEM) Sodium laureate powder samples were mounted on SEM stubs (32 mm) using a carbon sticky. The samples were sputter coated with platinum. The samples were loaded into the Hitachi SU5000 Scanning Electron Microscope. The samples were imaged in high vacuum mode and images were acquired using the secondary electron (SE) detector. The voltage was set to 2 kV and the Spot Intensity was set to 30 (unities). Images were acquired at several magnifications. Imaging in FIG. 9 demonstrated the morphology of the agglomerated crystals of sodium laureate existed as well-defined plate-like primary particles. Such a morphology is extremely difficult to achieve without spray drying and is desirable over elongates plates or needles because it tends to display superior compression performance in manufacturing procedures and dosage forms.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

### EQUIVALENTS

While several inventive embodiments have been described and illustrated herein, those of ordinary skill in the art will readily envision a variety of other means for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, material, and/or method described herein. In addition, any combination of two or more such features, materials, and/or methods, if such features, materials, and/or methods are not mutually inconsistent, is included within the scope of the disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

It is understood that, whenever a composition is disclosed or claimed as “comprising” one or more features, embodiments wherein such compositions “consist of” and “consist essentially of” these one or more features are likewise disclosed or claimed. The transitional phrase “consisting essentially of” shall have its ordinary meaning as used in the field of patent law. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Whereas the transitional phrases “consisting of and “consisting essentially of shall be closed or semi-closed transitional phrases, respectively, as set forth in MPEP 2111.03. It should also be understood that, unless clearly indicated to the contrary, in any methods claimed herein that include more than one step or act, the order of the steps or acts of the method is not necessarily limited to the order in which the steps or acts of the method are recited.

1. A process for preparing agglomerated crystals of a sodium salt of a medium chain fatty acid comprising the steps of:
a dissolving a medium chain fatty acid in a first solvent to produce a first solution, wherein the first solvent comprises an aprotic polar solvent;
b) adding to the first solution (i) a second solvent, wherein the second solvent comprises a medium chain aliphatic hydrocarbon solvent, and (ii) a solution comprising a sodium salt of a short chain alcohol to create a resulting slurry; and
c) isolating agglomerated crystals from the resulting slurry.

2. The process of claim 1, wherein the first solvent is selected from acetonitrile, DMF, DMAC, and NMP.

3. The process of claim 1, wherein the second solvent is selected from heptane and hexane.

4. The process of claim 1, wherein the medium chain fatty acid comprises capric acid, and the first solvent comprises acetonitrile.

5. A process for preparing agglomerated crystals of a sodium salt of a medium chain fatty acid comprising the steps of:
a) dissolving a medium chain fatty acid in acetonitrile to produce a first solution;
b) adding to the first solution heptane and about 1 molar equivalent of a solution comprising a sodium salt of a short chain alcohol to create a resulting slurry; and
c) isolating agglomerated crystals from the resulting slurry.

6. The process of claim 5, wherein, in step b, the heptane and the solution comprising a sodium salt of a short chain alcohol are added to the first solution at a temperature below about 40° C.

7. The process of claim 5, wherein in step b, the heptane is added over about 3.5 to about 4.5 hours at a controlled rate, with constant stirring.

8. The process of claim 5, wherein, after step b, the resulting slurry is stirred for at least one hour.

9. The process of claim 5, wherein, after step b, the resulting slurry is stirred for 15 hours to 25 hours.

10. A process for preparing agglomerated crystals of sodium caprate comprising the steps of:
a) dissolving capric acid in acetonitrile to produce a first solution;
b) adding to the first solution heptane and about 1 molar equivalent of a solution comprising sodium methoxide to create a resulting slurry; and
c) isolating agglomerated crystals of sodium caprate from the resulting slurry.

11. The process of claim 10, wherein, in step b, the heptane and the solution comprising sodium methoxide are added to the first solution at a temperature below about 40° C. to induce a liquid-liquid phase separation.

12. The process of claim 10, wherein, after step b, the resulting slurry is stirred for at least one hour.

13. The process of claim 10, wherein 6 L/kg to 8 L/kg of acetonitrile is added in step a.

14. The process of claim 10, wherein 1.7 L/kg to 2.1 L/kg of heptane is added in step b.

15. The process of claim 10, wherein in step b, the heptane is added over about 3.5 to about 4.5 hours.

16. The process of claim 10, comprising the steps of:
a) dissolving capric acid in 6 L/kg to 30 L/kg of acetonitrile to produce a first solution;
b) adding to the first solution 1.5 L/kg to 5 L/kg of heptane and 0.75 to 1.5 molar equivalent of a solution comprising about 20 to about 40 wt % of sodium methoxide at a temperature below about 40° C. to induce a liquid-liquid phase separation and create a resulting slurry;
c) stirring the resulting slurry for at least one hour; and
d) filtering the resulting slurry to provide agglomerated sodium caprate crystals.

17. The process of claim 10, comprising the steps of:
a) dissolving capric acid in 6 L/kg to 30 L/kg of acetonitrile, to produce a first solution;
b) adding to the first solution about 1 molar equivalent of a solution comprising about 30 wt % sodium methoxide, over about 4.5 to about 5.5 hours, at a temperature of about 20° C. to about 30° C., with constant stirring;
c) about 1 hour after adding the solution comprising sodium methoxide in step b, adding about 1.5 L/kg to about 4 L/kg of heptane to produce resulting slurry;
d) stirring the resulting slurry for 15 hours to 25 hours; and
e) filtering the resulting slurry to separate resulting solids and drying said resulting solids to provide agglomerated crystals of sodium caprate.

18. The process of claim 17, wherein in step c, the heptane is added over about 3.5 to about 4.5 hours at a controlled rate, with constant stirring.

19. A composition comprising a sodium salt of a medium chain fatty acid generated using the process of claim 1.

20. A composition comprising sodium caprate, wherein the composition exhibits a Carr's Index of less than 9.0%.

21. A composition comprising sodium caprate, wherein the composition exhibits a Hausner Ratio of less than 1.15.
